(19) United States
(12) Patent Application Publication
(10) Pub. No.: US 2026/0192281 A1
(43) Pub. Date: Jul. 9, 2026

(54) COMPOSITION AND METHODS FOR CLEANING

(71) Applicant: Genzyme Corporation, Cambridge, MA (US)

(72) Inventor: Joseph P. KUTZKO, Southborough, MA (US)

(73) Assignee: Genzyme Corporation, Cambridge, MA (US)

(21) Appl. No.: 19/128,491

(22) PCT Filed: Nov. 9, 2023
(86) PCT No.: PCT/US2023/079275

§ 371 Date: May 8, 2025

Related U.S. Application Data
(60) Provisional application No. 63/382,985, filed on Nov. 9, 2022.

(51) Int. Cl. B01J 20/34; B01D 15/20; B01D 15/32; B01J 20/26; C07K 1/20; C11D 3/04; C11D 3/20; C12N 9/24

(52) U.S. Cl. CPC B01J 20/3475; B01D 15/203; B01D 15/327; B01J 20/26; B01J 20/3425; C07K 1/20; C11D 3/044; C11D 3/2048; C12N 9/2402; C12Y 302/01045

(57) ABSTRACT

The present technology relates to a novel cleaning method for chromatography media and/or supporting equipment comprising treatment with a novel cleaning solution comprising sodium hydroxide and hexylene glycol.

### CROSS-REFERENCE TO RELATED APPLICATIONS

This application claims priority from U.S. Provisional Patent Application 63/382,985, filed Nov. 9, 2022, the disclosure of which is incorporated herein by reference in its entirety.

### BACKGROUND

The high commercial demand for biologics has led to pharmaceutical companies placing an emphasis on maximizing productivity and product quality whilst controlling costs associated with manufacturing. This push for improved product quality has allowed hydrophobic interaction chromatography (HIC) to rise to the forefront as a versatile technique for purifying analytes of interest (e.g., proteins) from crude mixtures while maintaining biological activity due to the use of conditions and matrices that operate under less denaturing conditions. HIC combines the nondenaturing characteristics of salt precipitation and the precision of chromatography to provide high resolution along with good recovery of biological activity. HIC is commonly used to separate a desired monomeric form of the target analyte from less desirable aggregated forms. When the target analyte is a protein, HIC may also provide superior selectivity for the removal of misfolded or truncated forms of the protein.

Any bioprocess chromatography application, including HIC, requires a high degree of control over contaminant and impurity removal. These contaminants include, but are not limited to, proteins, carbohydrates, lipids, lipopolysaccharides (e.g., endotoxins), lipoproteins, and/or nucleic acids. Macromolecular impurities, e.g., proteins, carbohydrates, lipids, nucleic acids, etc., are often addressed by taking advantage of various intermolecular forces and separating out the target analyte from impurities during wash and elution phases of the chromatography operation. Traditionally, HIC resin decontamination methods include treatment with cleaning solutions comprising sodium hydroxide, ethylene glycol, and ethanol. However, ethylene glycol is extremely toxic and ethanol is highly flammable.

### SUMMARY

The present disclosure provides a cleaning solution consisting essentially of a strong base and hexylene glycol. In some embodiments, the strong base is a hydroxide of an alkali metal. In some embodiments, the strong base is sodium hydroxide. In some embodiments, the concentration of sodium hydroxide is from about 0.1 M to about 0.5 M. In some embodiments, the concentration of hexylene glycol is from about 35% to about 55%. In some embodiments, the cleaning solution consists essentially of about 0.1 M sodium hydroxide and about 50% hexylene glycol. In some embodiments, the pH of the cleaning solution is about 12.

In some embodiments, a cleaning solution of the present disclosure is for use in a method for functionally cleaning chromatography media and/or supporting equipment.

In some embodiments, the present disclosure provides a method for functionally cleaning chromatography media and/or supporting equipment, comprising contacting the chromatography media and/or supporting equipment with a cleaning solution of the present disclosure.

In some embodiments, the chromatography media is a hydrophobic interaction chromatography (HIC) resin. In some embodiments, the HIC resin is selected from Capto MMC, Capto Butyl, Capto Phenyl, and Toyopearl Hexyl—650 C. In some embodiments, the HIC resin is Capto Butyl.

In some embodiments, the present disclosure provides a purification process comprising a cleaning step according to a cleaning method disclosed herein. In some embodiments, the cleaning step comprises a step within an integrated continuous biomanufacturing process for purification of a polypeptide. In some embodiments, the polypeptide is an antibody or a recombinant enzyme. In some embodiments, the recombinant enzyme is a human recombinant enzyme. In some embodiments, the human recombinant enzyme is β-glucocerebrosidase (β-D-glucosyl-N-acylsphingosine glucohydrolase). In some embodiments, the recombinant enzyme is Cerezyme®.

### BRIEF DESCRIPTION OF THE FIGURES

FIG. 1A depicts a HiScreen™ Capto™ Butyl column after cleaning with the standard cleaning solution comprising 0.1 M sodium hydroxide, 50% ethylene glycol, and 10% ethanol; FIG. 1B depicts a HiScreen™ Capto™ Butyl column after cleaning with a solution comprising 0.1 M sodium hydroxide and 50% hexylene glycol.

FIG. 2 is a chromatogram for screening various cleaning solutions—CIP 1 (100 mM sodium acetate, 700 mM arginine, pH 3); CIP 2 (1.0 M sodium hydroxide); CIP 3 (0.1 M sodium hydroxide, 50% ethylene glycol, and 10% ethanol); and CIP 4 (0.5 M sodium hydroxide and 50% hexylene glycol).

FIG. 3 is a plot depicting the change in specific activity (U/mg) of an exemplary human recombinant enzyme eluted from a Capto™ Butyl column over 40-cycles.

FIG. 4 is a plot depicting the change in activity yield (%) of an exemplary human recombinant enzyme eluted from a Capto™ Butyl column over 40-cycles.

### DETAILED DESCRIPTION

Features, objects, and advantages of the present technology are apparent in the detailed description that follows. It should be understood, however, that the detailed description, while indicating embodiments and aspects of the present technology, is given by way of illustration only, not limitation. Various changes and modifications within the scope of the present technology will become apparent to those skilled in the art from the detailed description.

The definitions of certain terms as used in this specification are provided below. Unless defined otherwise, all technical and scientific terms used herein generally have the same meaning as commonly understood by one of ordinary skill in the art to which this present technology belongs.

### I. Definitions

The moieties described below can be substituted or unsubstituted. “Substituted” refers to replacement of a hydrogen atom of a molecule or an R-group with one or more additional R-groups such as deuterium, halogen, alkyl, haloalkyl, alkenyl, alkoxy, alkoxyalkyl, alkylthio, trifluoromethyl, acyloxy, hydroxy, hydroxyalkyl, mercapto, carboxy, cyano, acyl, aryloxy, aryl, arylalkyl, heteroaryl, amino, aminoalkyl, alkylamino, dialkylamino, morpholino, piperidino, pyrrolidin-1-yl, piperazin-1-yl, nitro, phosphine, phosphinate, phosphonate, sulfate, =O, =S, or other R-groups. Unless otherwise indicated, an optionally substituted group may have a substituent at each substitutable position of a group. Combinations of substituents contemplated herein are preferably those that result in the formation of stable (e.g., not substantially altered for a week or longer when kept at a temperature of 40° C. or lower in the absence of moisture or other chemically reactive conditions), or chemically feasible, compounds.

Unless specified otherwise, the term “strong base” as used herein refers to a hydroxide of an alkali metal or an alkaline earth metal. Examples of strong base include, but are not limited to, sodium hydroxide (NaOH), potassium hydroxide (KOH), lithium hydroxide (LiOH), rubidium hydroxide (RbOH), cesium hydroxide (CsOH), calcium hydroxide (Ca(OH)₂), barium hydroxide (Ba(OH)₂), and strontium hydroxide (Sr(OH)₂).

Unless specified otherwise, the term “glycol” as used herein refers to a compound of formula (R²)(R³)—C(OH)—C(OH)—(R⁴)(R⁵), wherein R², R³, R⁴, and R⁵ are each, independently, hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio.

The term “functional cleaning” as used herein refers to a cleaning process wherein there is no measurable carryover of impurities (e.g., host cell proteins, protein aggregates, DNA, lipids, cell debris, etc.) from one purification cycle to the next, and/or wherein build-up of impurities is very low such that the lifetime of chromatographic media and/or supporting equipment will typically exceed 100 useful cycles.

The term “variant” as used herein encompasses any form of a particular protein that is recombinantly expressed in a host cell or non-native host cell. In some embodiments, the term “variant” refers to a protein recombinantly expressed from its native DNA sequence. In some embodiments, the term “variant” refers to a protein recombinantly expressed from a codon optimized DNA sequence. In some embodiments, the term “variant” refers to a recombinantly expressed full-length protein. In some embodiments, the term “variant” refers to a recombinantly expressed truncated form of the protein. In some embodiments, the term “variant” refers to a recombinantly expressed mutant protein. In some embodiments, the mutant protein contains point mutations at one or more positions in its amino acid sequence. In some embodiments, the term “variant” refers to a recombinantly expressed engineered protein, e.g., a genetically engineered protein. In some embodiments, the term “variant” refers to a recombinantly expressed artificial protein.

### II. Cleaning Solution

Provided herein, in some embodiments, are compositions and methods for cleaning chromatography media and/or supporting equipment for reuse. Chromatography reuse is a changeover procedure where the chromatography material is cleaned so that it can be reused to purify the same target analyte or a different target analyte. Significant cost savings can be achieved if a chromatography resin, e.g., a HIC resin, is reused for the purification of multiple analytes or multiple batches of the same analyte. In some embodiments, a cleaning solution of this disclosure is suitable for functional cleaning of chromatography media and/or supporting equipment such that there is no measurable carryover of impurities from one elution cycle to the next, or build-up of impurities is very low such that the column lifetime exceeds 100 useful cycles.

Conventional chromatography media can be cleaned and sanitized to sufficiently reduce tissue culture impurities (protein, DNA, lipid, cell debris, etc.) with a combination of sodium hydroxide and salt. Hydrophobic interaction and multimodal interaction chromatography typically require a solvent in addition to sodium hydroxide and salt to clean. In some embodiments, provided herein are compositions for cleaning chromatography media and/or supporting equipment (e.g., chromatography column, such as a hydrophobic interaction column (HIC)).

In some embodiments, the present technology relates to a cleaning solution comprising a strong base and a glycol.

In some embodiments, a cleaning solution of this disclosure comprising a strong base and a glycol (e.g., sodium hydroxide and a glycol) is efficient at eluting and cleaning HIC columns, for instance, HIC columns used in a process for preparing a recombinant enzyme.

In some embodiments, the present technology relates to a cleaning solution comprising sodium hydroxide. In some embodiments, the solution comprises about 0.05-0.5 M sodium hydroxide. In some embodiments, the solution comprises about 0.05 M, 0.08 M, 0.1 M, 0.15 M, 0.2 M, 0.25 M, 0.3 M, 0.35 M, 0.4 M, 0.45 M, or 0.5 M sodium hydroxide. In some embodiments, the solution comprises about 0.1 M sodium hydroxide.

In some embodiments, the present technology relates to a cleaning solution comprising hexylene glycol. In some embodiments, the solution comprises about 8-80% hexylene glycol. In some embodiments, the solution comprises about 10-70%, about 12-60%, about 14-50%, about 16-40%, about 18-30%, or about 20-25% hexylene glycol. In some embodiments, the solution comprises about 8%, 10%, 12%, 14%, 16%, 18%, 20%, 25%, 30%, 35%, 40%, 45%, 50%, 55%, 60%, 70%, or 80% hexylene glycol. In some embodiments, the solution comprises about 50% hexylene glycol.

In some embodiments, the present technology relates to a cleaning solution comprising sodium hydroxide and hexylene glycol. In some embodiments, a cleaning solution comprising sodium hydroxide and hexylene glycol is superior in cleaning and eluting a HIC column, compared to a cleaning solution comprising ethylene or propylene glycol and sodium hydroxide without ethanol or some other co-solvent. In some embodiments, when a cleaning solution comprising sodium hydroxide and hexylene glycol is used in a biomanufacturing process (e.g., an integrated continuous biomanufacturing process), product yield and peak sharpness of the target analyte, e.g., a recombinant protein (such as a recombinant enzyme), is improved.

In some embodiments, the present technology relates to a cleaning solution comprising about 0.05 M to 0.5 M sodium hydroxide and about 8-80% hexylene glycol. In some embodiments, the solution comprises about 0.05 M sodium hydroxide and 35-55% hexylene glycol, 0.08 M sodium hydroxide and 35-55% hexylene glycol, 0.1 M sodium hydroxide and 35-55% hexylene glycol, 0.15 M sodium hydroxide and 35-55% hexylene glycol, 0.2 M sodium hydroxide and 35-55% hexylene glycol, 0.25 M sodium hydroxide and 35-55% hexylene glycol, 0.3 M sodium hydroxide and 35-55% hexylene glycol, 0.35 M sodium hydroxide and 35-55% hexylene glycol, 0.4 M sodium hydroxide and 35-55% hexylene glycol, 0.45 M sodium hydroxide and 35-55% hexylene glycol, or 0.5 M sodium hydroxide and 35-55% hexylene glycol. In some embodiments, the solution comprises about 0.1 M sodium hydroxide and 50% hexylene glycol.

In some embodiments, the present technology relates to a cleaning solution comprising about 0.05 M to 0.5 M sodium hydroxide and about 50% hexylene glycol. In some embodiments, the solution comprises about 0.05 M sodium hydroxide and 50% hexylene glycol, 0.08 M sodium hydroxide and 50% hexylene glycol, 0.1 M sodium hydroxide and 50% hexylene glycol, 0.15 M sodium hydroxide and 50% hexylene glycol, 0.2 M sodium hydroxide and 50% hexylene glycol, 0.25 M sodium hydroxide and 50% hexylene glycol, 0.3 M sodium hydroxide and 50% hexylene glycol, 0.35 M sodium hydroxide and 50% hexylene glycol, 0.4 M sodium hydroxide and 50% hexylene glycol, 0.45 M sodium hydroxide and 50% hexylene glycol, or 0.5 M sodium hydroxide and 50% hexylene glycol. In some embodiments, the solution comprises about 0.1 M sodium hydroxide and 50% hexylene glycol.

In some embodiments, the present technology relates to a cleaning solution with a pH of about 12. In some embodiments, the solution has a pH of about 10, about 10.5, about 11, about 11.5, about 11.7, about 11.9, about 12.1, about 12.3, about 12.5, or about 13. In some embodiments, the solution has a pH of about 12.0.

In some embodiments, the cleaning solution comprises about 0.1 M sodium hydroxide and about 50% hexylene glycol and has a pH of about 12.0.

### III. Methods of Cleaning

In some embodiments, the present disclosure provides a cleaning method for chromatography media and/or supporting equipment comprising treatment with a cleaning solution comprising a strong base and a glycol. The potential contaminants addressed by the present technology include, without limitation, carryover impurities, protein DNA, lipid, cell debris, etc. In some embodiments, the present method provides functionally cleaned chromatography media and/or supporting equipment such that there is no measurable carryover of impurities from one purification cycle to the next, and/or build-up of impurities is very low such that the chromatography media and/or supporting equipment have extended lifespans.

In some embodiments, the present disclosure relates to a method of cleaning chromatographic media and/or supporting equipment by contacting the media and/or equipment with a cleaning solution described in the present disclosure. In some embodiments, the cleaning solution comprises a strong base and a glycol.

In some embodiments, the present technology relates to a method of cleaning chromatographic media and/or supporting equipment by contacting the media and/or equipment with a cleaning solution comprising sodium hydroxide. In some embodiments, the solution comprises about 0.05-0.5 M sodium hydroxide. In some embodiments, the solution comprises about 0.05 M, 0.08 M, 0.1 M, 0.15 M, 0.2 M, 0.25 M, 0.3 M, 0.35 M, 0.4 M, 0.45 M, or 0.5 M sodium hydroxide. In some embodiments, the solution comprises about 0.1 M sodium hydroxide.

In some embodiments, the present technology relates to a method of cleaning chromatographic media and/or supporting equipment by contacting the media and/or equipment with a cleaning solution comprising hexylene glycol. In some embodiments, the solution comprises about 8-80% hexylene glycol. In some embodiments, the solution comprises about 10-70%, about 12-60%, about 14-50%, about 16-40%, about 18-30%, or about 20-25% hexylene glycol. In some embodiments, the solution comprises about 8%, 10%, 12%, 14%, 16%, 18%, 20%, 25%, 30%, 35%, 40%, 45%, 50%, 55%, 60%, 70%, or 80% hexylene glycol. In some embodiments, the solution comprises about 50% hexylene glycol.

In some embodiments, the present technology relates to a method of cleaning chromatographic media and/or supporting equipment by contacting the media and/or equipment with a cleaning solution comprising about 0.05 M sodium hydroxide and 35-55% hexylene glycol, 0.08 M sodium hydroxide and 35-55% hexylene glycol, 0.1 M sodium hydroxide and 35-55% hexylene glycol, 0.15 M sodium hydroxide and 35-55% hexylene glycol, 0.2 M sodium hydroxide and 35-55% hexylene glycol, 0.25 M sodium hydroxide and 35-55% hexylene glycol, 0.3 M sodium hydroxide and 35-55% hexylene glycol, 0.35 M sodium hydroxide and 35-55% hexylene glycol, 0.4 M sodium hydroxide and 35-55% hexylene glycol, 0.45 M sodium hydroxide and 35-55% hexylene glycol, or 0.5 M sodium hydroxide and 35-55% hexylene glycol. In some embodiments, the solution comprises about 0.1 M sodium hydroxide and 35-55% hexylene glycol.

In some embodiments, the present technology relates to a method of cleaning chromatographic media and/or supporting equipment by contacting the media and/or equipment with a cleaning solution comprising about 0.05 M sodium hydroxide and 50% hexylene glycol, 0.08 M sodium hydroxide and 50% hexylene glycol, 0.1 M sodium hydroxide and 50% hexylene glycol, 0.15 M sodium hydroxide and 50% hexylene glycol, 0.2 M sodium hydroxide and 50% hexylene glycol, 0.25 M sodium hydroxide and 50% hexylene glycol, 0.3 M sodium hydroxide and 50% hexylene glycol, 0.35 M sodium hydroxide and 50% hexylene glycol, 0.4 M sodium hydroxide and 50% hexylene glycol, 0.45 M sodium hydroxide and 50% hexylene glycol, or 0.5 M sodium hydroxide and 50% hexylene glycol. In some embodiments, the cleaning solution comprises about 0.1 M sodium hydroxide and 50% hexylene glycol.

In some embodiments, the present technology relates to a method of cleaning chromatographic media and/or supporting equipment by contacting the media and/or equipment with a cleaning solution that has a pH of about 12. In some embodiments, the cleaning solution has a pH of about 10, about 10.5, about 11, about 11.5, about 11.7, about 11.9, about 12.1, about 12.3, about 12.5, or about 13. In some embodiments, the cleaning solution has a pH of about 12.0.

In some embodiments, the chromatographic media and/or supporting equipment is treated with a cleaning solution of the present disclosure at a temperature from about 0° C. to about 40° C. In some embodiments, the chromatographic media and/or supporting equipment is treated with the cleaning solution at a temperature from about 0° C. to about 25° C. In some embodiments, the chromatographic media and/or supporting equipment is treated with the cleaning solution at a temperature from about 5° C. to about 25° C. In some embodiments, the chromatographic media and/or supporting equipment is treated with the cleaning solution at a temperature from about 10° C. to about 25° C. In some embodiments, the temperature is about 5° C., about 10° C., about 15° C., about 20° C., about 22° C., about 25° C. about 30° C., about 35° C., or about 40° C. In some embodiments, the temperature is about 20° C. In some embodiments, the temperature is about 22° C.

In some embodiments, the present disclosure relates to a method of cleaning chromatographic media and/or supporting equipment by contacting the media and/or supporting equipment with a cleaning solution comprising about 0.1 M sodium hydroxide and about 50% hexylene glycol, about pH 12, and at a temperature of about 20-22° C.

By way of example, and not limitation, in some embodiments, chromatographic media refers to any material packed into a column. In some embodiments, the material is a resin or a particle. In some embodiments, the resin is a polymeric support or base matrix. In some embodiments, the polymeric support or the base matrix may comprise, without limitation, agarose, cellulose, sepharose, polymethacrylate, or polyvinylether. In some embodiments, the polymeric support or base matrix is coupled to a ligand. In some embodiments, the ligand is an affinity ligand or a hydrophobic ligand. In some embodiments, the hydrophobic ligand comprises an alkyl group (e.g., straight chain alkyl group such as butyl, octyl, etc.) or an aryl group (e.g., phenyl).

In some embodiments, the chromatographic media is designed for use in immobilized metal affinity chromatography (IMAC), ion exchange chromatography (IEX), e.g., cation exchange chromatography (CEX) or anion exchange chromatography (AEX), gel filtration chromatography (also known as size-exclusion chromatography (SEC)), hydrophobic interaction chromatography (HIC), supercritical fluid chromatography (SFC), high performance liquid chromatography (HPLC), ultra-high performance liquid chromatography (UHPLC), high turbulence liquid chromatography (HTLC), normal phase chromatography (NPC), reverse phase chromatography (RPC), capillary liquid chromatography, electrochromatography, membrane chromatography, monolith chromatography, and nano or capillary liquid chromatography.

By way of example, and not limitation, in some embodiments, supporting equipment is one or more selected from chromatographic columns, pumps, injectors, interconnecting tubing, detectors, sample collectors, mixers, flow restrictors, inline filters, valves, bubble traps, and all other liquid contact surfaces.

In some embodiments, the present technology does not impair the function of the chromatography media/resin or abridge the lifetime performance of the media/resin. Additionally, or alternatively, in some embodiments, a cleaning solution of the present disclosure has low toxicity, is non-flammable, and/or does not cause protein aggregation. In some embodiments, a cleaning solution of the present disclosure has high wettability, wherein the high wettability allows for efficient distribution throughout the chromatography media itself, i.e., chromatography beads and bead pores.

### IV. Sample Preparation

In some embodiments, the present technology can be applied toward the purification and/or detection of one or more analytes of interest from any source sample, such as biological samples or environmental samples. In some embodiments, the biological sample may be from humans, animals, plants, microorganisms, or any living organelles, such as cell and tissue cultures, tissue biopsy, whole blood, dry blood spot, plasma, de-proteinated plasma, serum, de-proteinated serum, ascites fluid, semen, sputum, urine, feces, perspiration, saliva, bile, tears, cerebrospinal fluid, swabs from body sites, skin, and hair. In some embodiments, the environmental sample may be an air sample, soil sample, water sample, food sample, and any material sample. In some embodiments, the source sample is obtained from cell/tissue cultures. In some embodiments, the source sample is obtained from cell/tissue culture supernatants. In some embodiments, the source sample is obtained from cell lysates.

In some embodiments, analytes of interest may be, for example, small molecules such as drug substances and macromolecules such as polypeptides, peptides, nucleic acids, lipids or fatty acids, carbohydrates, lipoproteins, lipopolysaccharides (e.g., endotoxins), hormones, vitamins, steroids, and metabolites. In some embodiments, the analyte of interest is a polypeptide. In some embodiments, the polypeptide is a therapeutic polypeptide. In some embodiments, the polypeptide is an enzyme or a recombinant enzyme. In some embodiments, the recombinant enzyme is a human recombinant enzyme. In some embodiments, the polypeptide is a non-enzymatic protein, e.g., a structural protein (e.g., collagen), a transport protein (e.g., hemoglobin), a regulatory protein (e.g., peptide hormones), a motor protein (e.g., myosin), or an immune protein (e.g., antibodies). In some embodiments, the polypeptide is an antibody, e.g., a monoclonal antibody (mAb), a polyclonal antibody (pAb), a bispecific antibody (BsAb), a trispecific antibody (TsAb), an antigen binding fragment thereof, or an antibody fusion protein. In some embodiments, the antibody is a recombinant monoclonal antibody. The term “antigen-binding fragment” as used herein refers to one or more fragments of an antibody that retain the ability to specifically bind to the same antigen as the whole antibody from which the portion is derived. Examples of “antigen-binding fragment” include, without limitation, a Fab fragment, a F(ab′)₂ fragment, a Fd fragment, a Fv fragment, a dAb fragment, an isolated complementarity determining region (CDR), scFv, and a diabody.

In some embodiments, the source sample (e.g., clarified cell culture fluid from a bioreactor harvest) is directly loaded onto the chromatography column without further adjustment (e.g., depth filtration, pH adjustment, etc.), thereby providing the purification process with continuous manufacturing capability.

In some embodiments, majority of contaminants and interfering materials are removed before applying the chromatography method to the source sample. In some embodiments, analytes of interest are enriched and isolated by filtration, precipitation, centrifugation, extraction, dilution, or a combination thereof. In some embodiments, analytes of interest are enriched from a source sample by solid phase extraction (SPE). SPE enriches analytes of interest by using sample preparation cartridges. The SPE extract containing the analytes may be dried and reconstituted in a solvent system compatible with the chromatography system.

In some embodiments, analytes of interest are extracted from a source sample by liquid-liquid extraction (LLE). LLE is used to separate analytes based on their relative solubilities in two immiscible or partially miscible liquids, usually a polar solvent like water and a non-polar organic solvent. The target analyte is first partitioned by a solvent, after which it is extracted, concentrated, and diluted.

In some embodiments, analytes of interest are extracted from a source sample by solid supported liquid-liquid extraction (SLE). In SLE, an aqueous solution of the source sample is loaded onto a support comprising of diatomaceous earth. Following sample absorption into the support, it is washed several times with an organic extraction solvent such as methyl tert-butyl ether. After the analyte of interest has been partitioned into the organic phase, it is concentrated by drying before being reconstituted in a solvent compatible for the chromatography system.

In some embodiments, wherein analytes of interest are proteins, they are enriched from the source sample by protein precipitation extraction (PPE). Protein precipitation methods may include desalting, isoelectric point precipitation, and organic solvent extraction. By way of example, the source sample is prepared for loading into chromatography system by desalting. This protein precipitation technique relies on the protein being “salted out” of the solution in response to increasing concentration of a neutral salt such as ammonium sulfate. In some embodiments, the source sample is prepared by isoelectric point precipitation; this method may be used to precipitate contaminant proteins, rather than the target protein. The isoelectric point (pI) is the pH at which the net primary charge of a protein becomes zero. For most proteins, the pI lies in the pH range of 4 to 6. In some embodiments, inorganic acids such as hydrochloric acid and sulfuric acid are used as precipitants. A potential disadvantage to isoelectric point precipitation is the irreversible denaturation caused by the inorganic acids.

### V. Chromatography

In some embodiments, once the source sample has been processed by, e.g., by centrifugation and/or filtration, the clarified, unadjusted sample is loaded onto the chromatography system, e.g., a liquid chromatography system.

Liquid chromatography (LC) is a process of selectively retaining one or more components of a fluid solution as the fluid solution (mobile phase) permeates through a column of a finely divided substance (stationary phase) by pumping action, pressure, and or gravity to accomplish diffusion in and through the pores of the chromatography media. The retention of selective components in the fluid solution by the stationary phase results from the higher affinities of the components for the stationary phase than for the mobile phase. In some embodiments, the liquid chromatography used is hydrophobic interaction chromatography (HIC), affinity chromatography (AC), ion exchange chromatography (IEX), size-exclusion chromatography (SEC), supercritical fluid chromatography (SFC), high performance liquid chromatography (HPLC), ultra-high performance liquid chromatography (UHPLC), high turbulence liquid chromatography (HTLC), normal phase chromatography (NPC), reverse phase chromatography (RPC), capillary liquid chromatography, electrochromatography, membrane chromatography, monolith chromatography, nano or capillary liquid chromatography. In some embodiments, the liquid chromatography system used in this technology is hydrophobic interaction chromatography (HIC).

In some embodiments, analytes of interest are retained by the stationary phase and subsequently eluted. In some embodiments, analytes of interest are flow through the stationary phase without being retained. In some embodiments, analytes in the eluate or in the effluent are be monitored by a variety of means, including UV, fluorescence, refractive index, light scattering, and electrical conductivity, based on retention time, peak intensity, and peak area. In some embodiments, further detailed analysis of the analytes is performed with techniques such as mass spectrometry.

In some embodiments, the LC solvents include, without limitation, water, methanol, ethanol, acetonitrile, trifluoroacetic acid, heptafluorobutyric acid, ether, hexane, hexylene glycol, propylene glycol, ethylene glycol, ethyl acetate, and an organic solvent such as hydrocarbon solvents (e.g., aliphatic and aromatic solvents), oxygenated solvents (e.g., alcohols, glycols, ketones, aldehydes, glycol ethers, esters, and glycol ether esters), and halogenated solvents (e.g., chlorinated and brominated hydrocarbons). In some embodiments, the LC solvents are buffered, and may contain various salts and buffering agents routinely used in the art, e.g., sodium hydroxide, sodium acetate, sodium phosphate, ammonium acetate, ammonium formate, ammonium bicarbonate, acetic acid, trifluoroacetic acid, formic acid, trimethylamine, triethylamine, etc. In some embodiments, the LC solvents also include detergents such as Tween, SDS, etc.

### VI. Hydrophobic Interaction Chromatography (HIC)

In some embodiments, the liquid chromatography used is hydrophobic interaction chromatography (HIC). HIC separates analytes according to differences in their surface hydrophobicity. HIC exploits a reversible interaction between hydrophobic analytes and immobilized hydrophobic ligands on HIC matrices. The interaction between the hydrophobic analyte and the HIC resin is greatly influenced by the salt concentration of the chromatography buffer. A high salt concentration enhances the interaction between the analyte and the HIC resin; lowering the salt concentration weakens the interaction. The main advantage of HIC is that it preserves the biological activity of the target analyte due to the use of conditions and matrices that operate under less denaturing conditions. HIC is primarily used for the purification of proteins. However, HIC can also be applied toward the separation of nucleic acids, viruses, cells, and carbohydrates. The retained analytes are eluted in increasing order of hydrophobicity.

The adsorption behavior of an analyte on a HIC resin is determined by the type of immobilized ligand. Generally, straight chain alkyl ligands demonstrate hydrophobic character while aryl ligands exhibit a mixed mode behavior where both aromatic and hydrophobic interactions are possible. It has been observed that the binding capacity of the HIC resin increases with an increase in the degree of substitution of the immobilized ligand. The most widely used HIC matrices are hydrophilic carbohydrates, e.g., cross-linked agarose and synthetic copolymer materials. The most commonly used hydrophobic ligands immobilized on HIC matrices include straight chain alkyl ligands (e.g., C4-C10 alkyl ligands such as butyl, octyl, etc.) and aryl ligands (e.g., phenyl).

In some embodiments, the cleaning solution and the methods of use disclosed herein comprise cleaning a resin of an HIC column. Examples of HIC resins include but are not limited to phenyl-, butyl-, octyl-SEPHAROSE, BUTYL-SEPHAROSE® 4 Fast Flow, PHENYL SEPHAROSE™ High Performance, PHENYL SEPHAROSE™ 6 Fast Flow (low sub), and PHENYL SEPHAROSE™ 6 Fast Flow (high sub).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to the manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. Although a number of documents are cited herein, this citation does not constitute an admission that any of these documents forms part of the common general knowledge in the art.

### EXAMPLES

In order that this technology may be better understood, the following examples are set forth. These examples are for purposes of illustration only and are not to be construed as limiting the scope of the technology in any manner.

### Example 1: Cleaning Solution Optimization

This Example describes a study for screening cleaning solutions for purifying a human recombinant enzyme captured by HIC.

### Methods

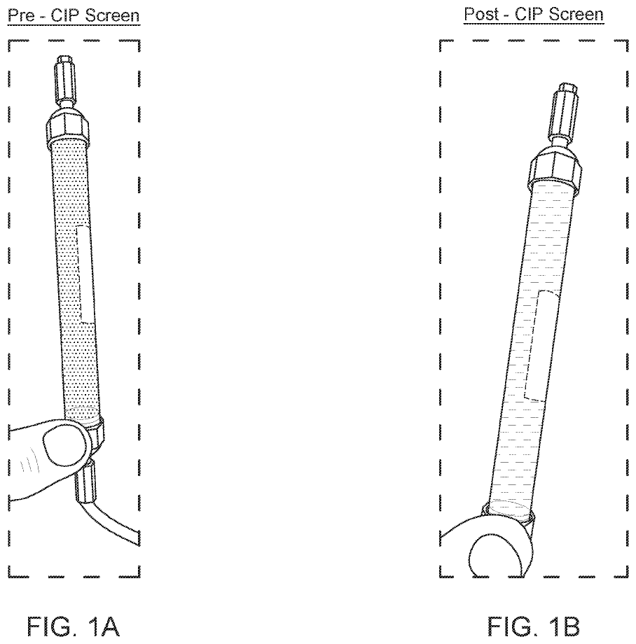

A HiScreen™ Capto™ Butyl column used for capturing a recombinant enzyme from clarified, unadjusted harvest was observed to have discoloration despite cleaning with a buffer comprising 0.1 M sodium hydroxide, 50% ethylene glycol, and 10% ethanol. After storing the column for three weeks in a storage buffer comprising 0.1 M sodium hydroxide, the rinse of the storage buffer showed an A280 absorbance above 3000 mAU. The presence of this peak, after storage, indicated that the column was not fully cleaned, alluding to the poor cleaning ability of the 0.1 M sodium hydroxide, 50% ethylene glycol, 10% ethanol solution. FIGS. 1A and 1B illustrate the discoloration of the HIC column prior to cleaning (pre-CIP) and regeneration to typical white resin following treatment with the optimized cleaning solution (post-CIP).

Several alternate CIP/cleaning solutions were screened through the HiScreen™ Capto™ Butyl column. A cleaning solution was selected based on the A280 absorbance of peaks generated during each cleaning phase. Use of each cleaning solution was bracketed by an equilibration buffer comprising 40 mM sodium phosphate, 150 mM NaCl, pH 6.7 to avoid any unexpected interactions between the different cleaning solutions and to isolate any UV peaks to a single cleaning solution. Table 1 summarizes the CIP screen run on the Capto™ Butyl column.

TABLE 1
Cleaning Solution Screening Procedure

| | | Volume | Flow | Flow rate |
|---|---|---|---|---|
| Step | Description | (CV) | direction | (cm/h) |
| EQ | 40 mM Sodium Phosphate, 150 mM Sodium | 5 | Up | 100 |
| Rinse | Chloride, pH 6.7 | | | |
| CIP 1 | 100 mM Acetate, 700 mM Arginine, pH 3 | 5 | Up | 30 |
| EQ | 40 mM Sodium Phosphate, 150 mM Sodium | 5 | Up | 100 |
| Rinse | Chloride, pH 6.7 | | | |
| CIP 2 | 1.0M Sodium Hydroxide | 3 | Up | 30 |

1 Hour Hold

| | | | | |
|---|---|---|---|---|
| RODI | Reverse Osmosis Deionized Water | 5 | Up | 100 |
| EQ | 40 mM Sodium Phosphate, 150 mM Sodium | 5 | Up | 100 |
| Rinse | Chloride, pH 6.7 | | | |
| CIP 3 | 10% Ethanol, 0.1M Sodium Hydroxide, 50% | 5 | Up | 30 |
| | Ethylene Glycol | | | |
| EQ | 40 mM Sodium Phosphate, 150 mM Sodium | 5 | Up | 100 |
| Rinse | Chloride, pH 6.7 | | | |
| CIP 4 | 50% Hexylene Glycol, 0.5M Sodium Hydroxide | 5 | Up | 30 |
| Storage | 0.1M Sodium Hydroxide | 5 | Up | 100 |

### Results

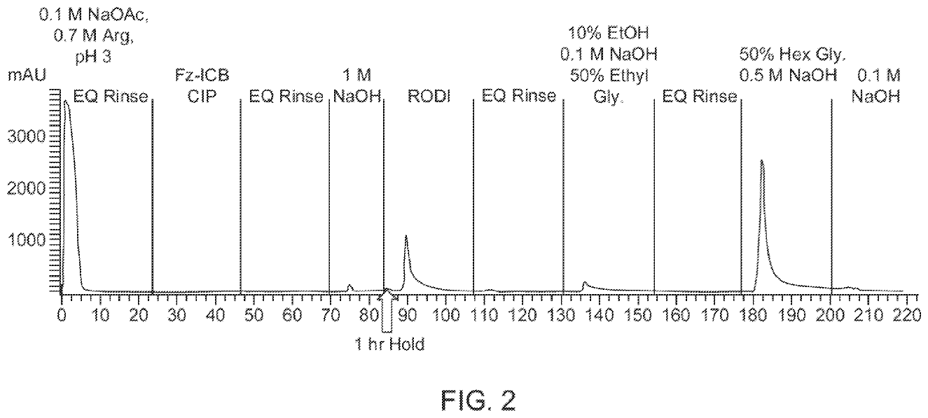

The chromatogram used to determine relative effectiveness of the screened cleaning solutions is shown in FIG. 2. The CIP 2 solution comprising 1.0 M sodium hydroxide had a minor absorbance peak, but was much more effective post the 1-hour hold rinse with reverse osmosis deionized (RODI) water. The CIP 3 solution comprising 0.1 M sodium hydroxide, 50% ethylene glycol, and 10% ethanol stripped a minor peak, but most of the impurities were still retained to the column as illustrated by the size of the A280 peak following treatment with the CIP 4 solution. The peak after cleaning with the CIP 4 solution comprising 50% hexylene glycol and 0.5 M sodium hydroxide was also near 3000 mAU and post this phase, the resin appeared visually clean as shown in FIG. 1B.

The standard cleaning solution for HIC columns comprising 0.1 M sodium hydroxide, 50% ethylene glycol, and 10% ethanol (CIP 3) produced a modest absorbance peak during the CIP screen. The CIP 4 cleaning solution comprising 50% hexylene glycol and 0.5 M sodium hydroxide significantly stripped the column and resulted in a visually cleaner appearing resin (FIG. 1B). Since 0.1 M sodium hydroxide appeared to lower the conductivity of the cleaning solution and thus improve the hydrophobicity of the solution, a cleaning solution comprising 0.1 M sodium hydroxide and 50% hexylene glycol solution was selected for further analysis.

### Example 2: Capto Butyl Cycling Study

### Methods

After optimizing the column cleaning protocol, a 40-run cycling study was conducted to determine the impact of run number on the quality and activity yield of the target protein.

### Results

A 40-cycle study was completed using daily collected harvest. Although all samples were initially tested for activity and high molecular weight (HMW) species, samples were only intermittently tested after Run 10. Table 2 summarizes the average activity and HMW results for the samples tested in the study. Eluates averaged 1.2 CV in volume with a standard deviation of 0.2 CV, indicating that a narrow elution peak can be expected throughout 40 cycles at scale.

TABLE 2
Summary of 40 Cycle performance for tested samples

| | Activity | Specific Activity | Activity Yield | HMW | |
|---|---|---|---|---|---|
| | (U/ml) | (U/mg) | (%) | (%) | CV |
| AVERAGE | 40.8 | 33.2 | 90.2 | 5.2 | 1.2 |
| STD DEV | 10.5 | 1.4 | 10.3 | 3.1 | 0.2 |

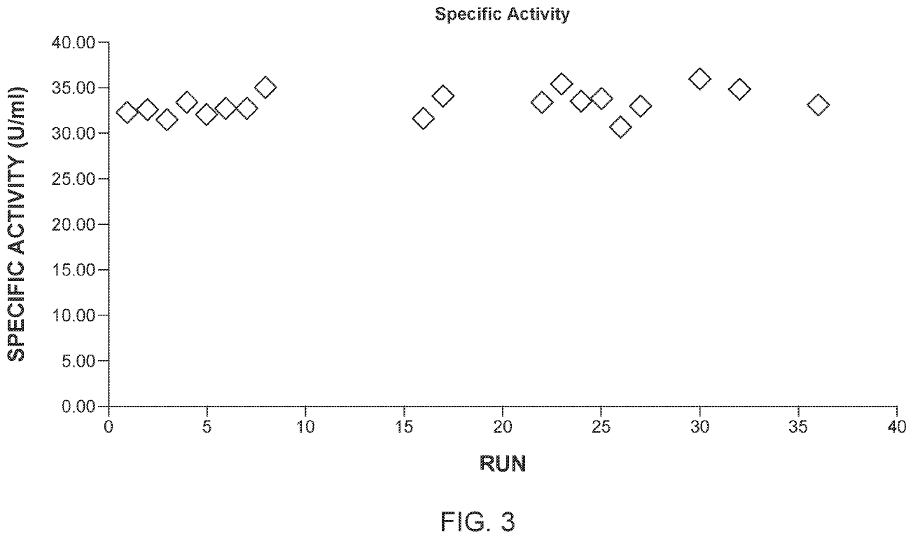

The average specific activity of the eluates was 33.2 U/mg, which is a 22% increase in purity relative to the Capto™ MMC average value of 27 U/mg. Specific activity of the eluate pools remained stable during the course of the study. The final run tested was 33.2 U/mg. FIG. 3 shows the specific activity of the target recombinant enzyme over the course of the study. Overall, the specific activity was robust with only a 1.4 U/mg standard deviation.

HMW species averaged 5.2% with a high standard deviation of 3.1%. However, HMW did not trend with cycle number and instead the highest HMW samples were observed in the middle of the reactor run.

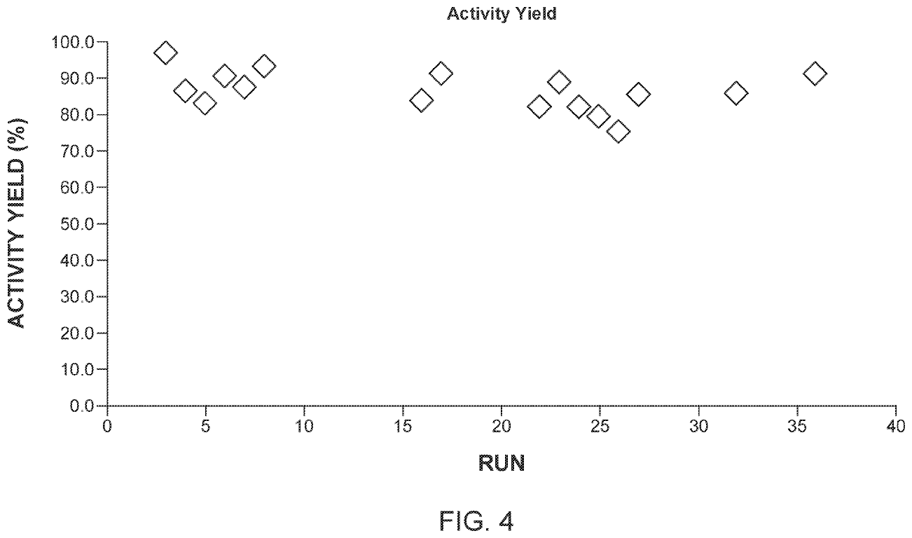

Activity yield varied significantly over the course of the study. The average specific activity yield was 90%, but the standard deviation for samples tested was 10.3%. FIG. 4 shows the activity yield over the course of 40 cycles. The activity assay variability was expected to be +/−5%, which could have contributed to the high standard deviation observed over the course of the study. Additionally, multiple runs with the same harvest pool were performed in a day and activity loss over the course of the load may have contributed to lower yields. Late run cycles still tested for activity yields +90% and therefore no significant reduction in yield was observed.

Blank runs were submitted for ATTO-Tag testing. The post 40 cycle blank was assumed to be the worst-case sample and most likely to contain protein carryover. None of the runs tested contained significant protein carryover (data not shown). The samples were above the detection limit of the assay but below accurate quantitation. None of the earlier blank runs were tested for protein carryover.

The cleaning solution of 50% hexylene glycol and 0.1 M sodium hydroxide not only provided excellent cleaning, but also did not negatively impact the resin performance over 40 cycles. The average specific activity of the samples was 33.1 U/mg with an activity yield of 90.1%. Furthermore, ATTO-tag testing indicated that no significant protein carryover existed for the late cycle runs.

1. A cleaning solution consisting essentially of a strong base and hexylene glycol.

2. The cleaning solution of claim 1, wherein the strong base is a hydroxide of an alkali metal.

3. The cleaning solution of claim 2, wherein the strong base is sodium hydroxide.

4. The cleaning solution of claim 3, wherein the concentration of sodium hydroxide is from about 0.1 M to about 0.5 M.

5. The cleaning solution of any one of claims 1 to 4, wherein the concentration of hexylene glycol is from about 35% to about 55%.

6. The cleaning solution of claim 1, consisting essentially of about 0.1 M sodium hydroxide and about 50% hexylene glycol.

7. The cleaning solution of any one of claims 1 to 6, wherein the pH of the solution is about 12.

8. The cleaning solution of any one of claims 1 to 7, for use in a method for functionally cleaning chromatography media and/or supporting equipment.

9. A method for functionally cleaning chromatography media and/or supporting equipment, comprising contacting the chromatography media and/or supporting equipment with a cleaning solution comprising a strong base and hexylene glycol.

10. The method of claim 9, wherein the strong base is a hydroxide of an alkali metal.

11. The method of claim 10, wherein the strong base is sodium hydroxide.

12. The method of claim 11, wherein the concentration of sodium hydroxide is from about 0.1 M to about 0.5 M.

13. The method of any one of claims 9 to 12, wherein the concentration of hexylene glycol is about 50%.

14. The method of claim 9, wherein the solution comprises about 0.1 M sodium hydroxide and about 50% hexylene glycol.

15. The method of any one of claims 9 to 14, wherein the pH of the solution is about 12.

16. The method of any one of claims 9 to 15, wherein the chromatography media is a hydrophobic interaction chromatography (HIC) resin.

17. The method of claim 16, wherein the HIC resin is selected from Capto MMC, Capto Butyl, Capto Phenyl, and Toyopearl Hexyl—650 C.

18. The method of claim 17, wherein the HIC resin is Capto Butyl.

19. A purification process comprising a cleaning step according to the method of any one of claims 9-18.

20. The purification process of claim 19, wherein the cleaning step comprises a step within an integrated continuous biomanufacturing process for purification of a polypeptide.

21. The purification process of claim 19 or 20, wherein the polypeptide is an antibody or a recombinant enzyme.

22. The purification process of claim 21, wherein the recombinant enzyme is a human recombinant enzyme.

23. The purification process of claim 22, wherein the human recombinant enzyme is β-glucocerebrosidase (β-D-glucosyl-N-acylsphingosine glucohydrolase).

24. The purification process of claim 21, wherein the recombinant enzyme is Cerezyme®.
